(19) United States
(12) Patent Application Publication
(10) Pub. No.: US 2003/0144271 A1
(43) Pub. Date: Jul. 31, 2003

(54) Methods for the treatment of substance abuse

(76) Inventor: Albert Shulman, Victoria (AU)

Correspondence Address: KNOBBE MARTENS OLSON & BEAR LLP, 2040 MAIN STREET, FOURTEENTH FLOOR, IRVINE, CA, 92614, US

(21) Appl. No.: 10/181,990

(22) PCT Filed: Jan. 22, 2001
(86) PCT No.: PCT/AU01/00060

(30) Foreign Application Priority Data
Jan. 22, 2000 (GB) 0001390.4
Jan. 26, 2000 (GB) 0001647.7
Dec. 21, 2000 (AU) PR 2237

(51) Int. Cl. A61K 31/55; A61K 31/485; A61K 31/455

(52) U.S. Cl. 514/214.03; 514/282; 514/355

(57) ABSTRACT

The present invention relates to methods of therapy for substance addiction comprising the administration to a subject in need thereof a combination of: (i) a μ-opioid receptor antagonist; (ii) a calcium channel blocker which is long-acting or in sustained-release form or which is nimodipine in rapid release form; and (iii) an NMDA glutamate receptor modulator; as well as combinations, kits and composition useful therefor.

### FIELD OF THE INVENTION

The present invention relates generally to methods of therapy and the combinations and compositions suitable therefor. In particular, the invention relates to methods for the treatment of substance abuse, including alcohol and opiate addiction.

### BACKGROUND OF THE INVENTION

Substance addiction involves an overwhelming and uncontrollable physical or psychological craving for a particular substance such as alcohol, heroin, morphine, methadone, nicotine, amphetamines, solvent inhalants, cocaine or marijuana and can impose significant social and financial costs to both the addict and the community. In many cases, withdrawal from the substance without adjunctive treatment, either pharmacological or psychotherapeutic, can cause acute physical and mental illness. Attempts to treat substance addiction by methods such as psychotherapy, behaviour modification or certain other drugs has had only limited success, with many addicts relapsing to chronic use. It is well known that the powerful and long-acting competitive and specific CNS μ, δ, κ-opioid receptor antagonist naltrexone is used for rapid opiate detoxification, but the method is not without hazard. Since the chronic addicting action of ethanol also appears to be mediated substantially, either directly at CNS opioid receptors or indirectly by liberation of endogenous opiates, long-term daily naltrexone treatment has also been used successfully to prevent craving and relapse in both detoxified opiate and ethanol addicts. More recently, the ethanol anti-craving drug acamprosate has enjoyed similar success in the long-term management of detoxified chronic ethanol addicts. However when given alone, neither naltrexone nor acamprosate provide an effective long-term cure for these conditions, relapse to chronic opiate or ethanol craving and dependence frequently recurring within 3-12 months.

It has now been found that a combined treatment using a μ-opioid receptor antagonist (μORA), an N-methyl-D-aspartic acid (NMDA) glutamate receptor modulator and a calcium channel blocker (CCB), which is either long-acting or formulated in a sustained-release form, may be effective in the treatment of substance abuse and may provide a longer-term remission or cure of, or reduction in, craving for, or relapse to chronic dependence on substances such as opiates, ethanol (alcohol) and other addicting drugs.

### SUMMARY OF THE INVENTION

Accordingly in a first aspect, the present invention provides a method of treating substance addiction in a subject in need thereof, which method comprises administering to said subject a combination of:

(i) a μ-opioid receptor antagonist;

(ii) a calcium channel blocker which is long-acting or in sustained-release form, or which is nimodipine in rapid release form, and

(iii) an NMDA glutamate receptor modulator.

Use of a μORA in the preparation of a medicament for the treatment of substance addiction, wherein said medicament is adapted for administration to a subject in combination with an NMDA glutamate receptor modulator and a CCB, which is long-acting or in sustained-release form or which is nimodipine in rapid release form.

Use of a CCB which is long-acting or in sustained-release form, or which is nimodipine in rapid release form, in the preparation of a medicament for the treatment of substance addiction, wherein said medicament is adapted for administration in combination with a μORA and an NMDA glutamate receptor modulator.

Use of an NMDA glutamate receptor modulator in the preparation of a medicament for the treatment of substance addiction where said medicament is adapted for administration in combination with a CCB, which is long-acting or in sustained-release form, or which is nimodipine in rapid release form, and a μORA.

Use of a μORA, an NMDA glutamate receptor modulator and a CCB in the preparation of a medicament for the treatment of substance addiction, wherein the CCB is long-acting or formulated in the medicament such that it is in a sustained-release form or which is nimodipine in rapid release form.

The invention also provides compositions containing at least two of a μORA, an NMDA glutamate receptor modulator and a CCB which is long lasting or in a sustained-release form or which is nimodipine in rapid release form.

In another aspect, the invention provides a kit comprising at least two of, preferably all of a μORA, an NMDA glutamate receptor modulator and a CCB which is long-acting or in sustained-release from or which is nimodipine in rapid release form, wherein said kit is in compartmentalized form adapted for the simultaneous or sequential administration of the at least 2 or all 3 of the μORA, NMDA glutamate receptor modulator and CCB.

### DESCRIPTION OF THE PREFERRED EMBODIMENTS

Without intending to limit the invention by theory, it is believed that the advantage of the combination of a μORA (such as naltrexone) an NMDA glutamate receptor, modulator (such as acamprosate) and a CCB is as follows:

The mesolimbic system appears to be the reward area of the brain that is primarily involved in the addictive potential of each of the following types of major drugs of dependence that are commonly abused by humans. For example:

1. the opiates such as heroin, codeine and morphine and derivatives such as pethidine and methadone. Methadone, because of its very long half-life, is also frequently prescribed legally as a helpful controlled alternative to decrease or prevent the use of an illicit opiate such as heroin.

2. the sedative-hypnotics ethanol, the barbiturates pentobarbitone and quinalbarbitone, and the benzodiazepines temazepam, diazepam and flunitrazepam.

3. the psychostimulants amphetamine and cocaine, and

4. the chemically distinct drugs nicotine, marijuana and phenyclidene.

Each of these drugs appears to produce its addictive rewarding euphoria in humans by promoting the release of the excitatory CNS neurotransmitter dopamine (DA) from the midbrain ventral tegmental area (VTA) for action in the mesolimbic nucleus accumbens (NAC). The mesolimbic excitatory dopaminergic neurotransmitter system originates in the VTA which projects its efferent dopamine-secreting nerve terminals to the functionally-related NAC.

It is considered that the addicting action of the above-listed drugs of abuse stems primarily from the following events that occur in the VTA and NAC. Using morphine as an example, these events involve the functionally-integrated, actions of four primary CNS neurotransmitter systems (presynaptic inhibitory GABA_{A}ergic, presynaptice inhibitory opoidergic, calcium-dependent excitatory dopaminergic and calcium-dependent excitatory glutamatergic), each resident in the VTA, as follows:

(i) under normal conditions, the opioidergic and dopaminergic systems show only physiological activity, the former under the modulatory control of the endogenous opioid neurotransmitter β-endorphin acting on μ opioid receptors, and the latter under the tonic inhibitory control of the GABA_{A}ergic system. Injection of the GABA_{A}ergic antagonists picrotoxin and bicucculine into the VTA increases the activity of the dopaminergic neurones, indicating the primary involvement of the GABA_{A}ergic inhibitory system.

(ii) VTA dopaminergic neurones do not appear to contain opioid receptors and do not respond directly to opiates.

(iii) morphine activates the inhibitory opioidergic system which suppresses the action of (disinhibits) the tonic inhibitory GABA_{A}ergic control. The resulting morphine-evoked GABA_{A}ergic disinhibition leads to activation of the excitatory dopaminergic system which results in increased release of DA from the VTA and its action in the NAC, manifesting in humans as a feeling of rewarding euphoria. Such increased DA release and action are blocked by perfusion of the opioid receptor antagonist naloxone into the VTA/NAC indicating the primary importance of the opioidergic system.

(iv) The other aforementioned major addicting drugs appear to produce their rewarding effects in a similar manner by primary action on these and possibly, in some cases, on associated neurotransmitter systems (eg, glutamatergic, cholinergic, serotonergic, neuropeptidergic) also relayed to the VTA from other parts of the brain.

The long-acting non-specific μ, δ, κ-opioid receptor antagonist, naltrexone, the ethanol anti-craving drug acamprosate and the CCBs act by different mechanisms on different neurotransmitter components of this primary functionally-integrated VTA neurotransmitter system. Naltrexone powerfully blocks VTA μ opioid receptors thereby preventing morphine activation of the inhibitory opioidergic system. Thus naltrexone restores GABA_{A}ergic inhibition of DA release and action in the NAC, and thereby terminates the maintenance of morphine-evoked rewarding eurphoria. In addition, naltrexone rapidly precipitates the opiate withdrawal syndrome which also leads to significant depression of DA release and action in the NAC, accompanied in humans by dysphoria and anxiety similar to the acute aversive effects produced by κ opioid ligands which also depress DA release and action in the NAC in rodents. Since naltrexone is also a powerful blocker of related CNS δ and κ opioid receptors it will similarly block δ and κ opioid receptor-regulated events which may also contribute to the opiate, ethanol and related addicting processes.

Acamprosate inhibits the adaptive increase in CNS voltage-operated L-type calcium channels associated with chronic ethanol dependence, and reduces calcium flux through these channels. It also inhibits the facilitating calcium-dependent NMDA glutamate receptor mediated excitatory glutamatergic neurotransmission in the VTA/NAC during ethanol withdrawal. Acamprosate may also restore the normal inhibitory activity of GABA_{A}ergic neurotransmission depressed by chronic ethanol intake.

On the other hand, CCB's appear to prevent the rewarding actions of opiates, ethanol and other addicting drugs by blocking VTA- and NAC-responsive voltage-operated presynaptic and postsynaptic L-type calcium channels. Such blockade would inhibit the morphine-evoked calcium-dependent presynaptic release of DA from the VTA and the postsynaptic rewarding action of DA released in the NAC.

Similar CCB-evoked inhibition may also occur in functionallly-related excitatory dopaminergic components of the brain's reward system such as the amygdala, hippocampus and the medial pre-frontal cortex associated with memory, emotion and psychological activity.

The large adaptive increase in CNS voltage-operated L-type calcium channels evoked by chronic opiate or ethanol dependence in rodents and humans may represent a further. example of a general pharmacological phenomenon, broadly termed supersensitivity, produced by a large diversity of blocking drugs in a variety of tissues with central or peripheral innervation in both vertebrates and invertebrates. For example, repeated morphine administration is known to produce chronic opiate dependence in rodents and humans, and to block neurotransmitter release and promote, its accumulation in a variety of responsive presynaptic nerve terminals in both the central and peripheral nervous systems. This generally results in a large compensatory increase in the number of neurotransmitter-sensitive postsynaptic receptors (supersensitivity), apparently manifesting in chronic opiate- or ethanol-dependent rodents and humans as CNS voltage-operated L-type neuronal calcium channels which are also involved in both withdrawal syndromes since they are blocked by structurally-diverse CCBs.

Direct experimental evidence is available which supports such chronic morphine-induced calcium-related presynaptic accumulation of calcium and neurotransmitter in rodent brain, and the operation of calcium-related supersensitivity in both withdrawn barbitone-dependent rats and removed model rat atrial preparations.

It seems likely that the wide-ranging symptoms and signs of opiate, ethanol and indeed barbiturate withdrawal may reflect the initial explosive then progressively decreasing release of large quantities of presynaptically-accumulated neurotransmitter(s) which hyperactivate their corresponding greatly increased number of neurotransmitter-sensitive compensatory supersensitive postsynaptic receptors. It also seems likely that such a process may involve primarily excitatory dopaminergic transmission and voltage-operated L-type calcium channels in the VTA/NAC and related limbic system and pre-frontal cortex. However, it may also occur at many and varied drug-responsive central and peripheral excitatory and inhibitory synapses distributed throughout the neuraxis.

Such events could explain the multiple uncontrolled motor, sensory and autonomic effects which comprise the opiate, ethanol, barbiturate and possibly related types of withdrawal syndromes seen peripherally, as well as the ensuing sub-optimal VTA/NAC dopaminergic function (and those of other involved CNS and PNS neurotransmitter systems) until physiological neurotransmission has been restored, following termination of withdrawal and a fall in the CNS addicting drug concentration to a sub-activating level.

Craving for drug-induced reward or reinforcement is evoked by detoxification from chronic dependence on opiates, ethanol or related addicting drugs. Craving may occur during or following such drug withdrawal and may signify, at least in part, the temporary sub-optimal VTA release and action of DA in the NAC until their return to physiological levels.

Thus, craving may involve:

1. addicting drug withdrawal-evoked depression of VTA DA release and action in the NAC induced by opiates, ethanol and related addicting drugs,

2. addicting drug withdrawal-induced activation of local dynorphin-regulated inhibitory κ opioid receptors in the NAC which would also decrease DA action in the NAC, and produce associated dysphoria and aversion. This would also tend to promote craving.

3. the associated addicting drug involvement of glutamatergic, GABA_{A}ergic, serotonergic, dopaminergic auto-inhibitory, and opioidergic and non-opioidergic neuropeptide neurotransmitter systems considered to be functionally integrated with the controlling excitatory dopaminergic transmitter system in the VTA/NAC. addicting drug-responsive calcium-dependent mechanisms in the VTA, NAC and functionally-related components of the brain's reward system such as amygdala, hippocampus and medial pre-frontal cortex. This seems likely from the results of a recent clinical in-patient pilot study involving selected CCB-evoked detoxification of chronic addicts dependent on opiates, ethanol, amphetamine, temazepam or marijuana.

It is postulated that naltrexone, acamprosate and the CCBs act on the same key functionally-integrated VTA/NAC multi-neurotransmitter system concerned primarily in mediating the rewarding and adverse effects of commonly abused major addicting drugs, although naltrexone, acamprosate and the CCBs appear to do so by different mechanisms involving different components of this same system.

It is now proposed that the combinations of the invention may display beneficial additive or synergistic effects in the prophylactic management of craving and/or relapse and in the enhancement of abstinence in detoxified addicts chronically dependent on opiates or ethanol, or on other addicting drugs discussed herein, each of which appears to involve similar calcium-related CNS processes.

Any compound which is capable of long-term blockade of μ-opioid receptors in the brain may be useful in the present invention. Suitable μORAs include naltrexone, nalmefine, buprenorphine and 1-α-acetylmethadol (LAAM). It will be understood that although the μORA must be capable of binding to and blocking the μ-opioid receptors in the brain, it may also be capable of binding to and blocking the other opioid receptors such as δ- and κ-opioid receptors. A preferred μORA for use in the present invention is naltrexone which non-specifically blocks μ,δ, and κ-opioid receptors in the CNS.

Compounds which induce modulation of the NMDA glutamate receptors include: CCP (3-(carboxypiperazin-4-yl)-propyl-1-phosphonic acid), dizocilpine (MK801), HA966 (3-amino-1-hydroxy-2-pyrrolidone), ibogaine, memantine, ifenprodil, eliprodil and acamprosate. A preferred NMDA glutamate receptor modulator is acamprosate.

The CCBs used in the present invention are either inherently long-acting (eg. amlodipine, nitrendipine, lacidipine or nisoldipine) or are formulated as a sustained-release preparation. “Long-acting” preferably refers to a compound which, like amlodipine, has a half life of 30-50 hours and takes some 5-10 days to reach a plateau plasma concentration. A “sustained-release” preparation is one in which the active ingredient is slowly released within the body once administered and maintains the desired drug concentration over a minimum period of time. A preferred sustained-release CCB preparation releases the active ingredient over a period of some 24 hours and maintains a plateau concentration that avoids or minimizes acute reflex cardio-acceleration frequently manifesting as palpitations, dizziness, headaches and flushing. Such plateau concentrations may depend on the general health, age and weight of the patient and can be readily determined by those skilled in the art or the attending physician. A preferred sustained-release CCB preparation maintains the desired plasma concentration for at least 18 and preferably 24 hours, thereby requiring only once daily administration to the patient. However, while the CCBs are generally administered in a long-acting or sustained-release form, it is possible to administer nimodipine in a: sustained-release or rapid-release form. A rapid-release form includes a native form which is rapidly solvated by water and rapidly released and absorbed. A rapid-release form may provide the desired plasma concentration for about 8 hours or less. It will be understood that a rapid-release form may require more than a once daily administration such as three or four times daily.

Examples of CCBs which may be suitable for use in the present invention include:

(i) The dihydropyridine compounds, which act at voltage-operated L-type calcium channels, such as nifedipine, nimodipine, nisoldipine, felodipine, amlodipine, darodipine, floridipine, lacidipine, isradipine, niguldipine, niludipine, oxadipine, elgodipine, riodipine, nilvadipine, lemdipine, nitrendipine, nicardipine.

(ii) Phenylalkylamine CCBs such as verapamil, gallopamil, anipamil, tiapamil or levemopamil, which also acts at L-type channels.

(iii) Benzothiazepine CCBs such as diltiazem or clentiazem, which also act at L-type calcium channels.

(iv) CCBs which act at other types of voltage operated calcium channels:

(a) voltage-operated T-type calcium channels: CCBs such as mibefradil (eg 50-100 mg daily) or flunarizine (eg. in divided doses from 10 to 100 mg daily). The dihydropyridine CCBs sustained-release felodipine (eg. 5-10 mg, once daily) and nicardipine (eg. 15-60 mg once or twice daily) also block these channels in a less selective manner.

(b) voltage-operated N-type calcium channels: blocked selectively and potently by a variety of marine snail (genus Conus) ω-conopeptide venoms, eg. the recently synthesized ω-conotoxin MVIIA/GVIA (SNX-111). This produces potent analgesia by inhibiting the calcium-dependent release of diverse excitatory CNS and PNS neurotransmitters at these N-type channels. The predominantly L-type CCBs nimodipine and nitrendipine (see above) also block these channels, but less selectively. This also applies to the non-competitive NMDA glutamate receptor antagonists ifenprodil and eliprodil, and aminoglycoside antibiotics such as gentamycin and tobramycin.

(c) voltage-operated P-type calcium channels: these CNS and PNS channels are selectively and potently blocked by the synthetic peptide venom ω-conotoxin MVIIC (SNX-230) and the funnel web spider venom ω-agatoxin IVA (AGAIVA). They are also blocked by the medicinal herb constituents eudesmol and daurisoline.

(d) voltage-operated P- Q- and O-type calcium channels: these and N-type CNS and PNS calcium channels are also blocked non-selectively by the synthetic co-conotoxin MVIIC (SNX 230) and by aminoglycoside antibiotics such as gentamycin and tobramycin.

(v) Non-selective calcium channel modulators which appear as well to block sodium and/or potassium-realted ion channels eg. flunarizine (for example in divided closes of from 10-100 mg daily) prenylamine (eg 30-60 mg thrice daily) or long-acting bepridil (eg 200-400 mg once daily).

Preferred CCBs for use in the present invention usually act on CNS voltage-operated L-type calcium channels and include: verapamil, nifedipine, amlodipine, felodipine, nitrendipine, nicardipine, nisoldipine, lacidipine, diltazem, nimodipine, isradipine and flunarizine, as well as possibly others mentioned in (i)-(iii), (iv)(b) and (v) above.

In addition, the combinations of the invention may further include drugs which act as, blockers at calcium-selective receptor-gated channels such as:

(a) α-adreno-, CB1/2 cannabinoid-and μ/κ opioid-linked receptors and calcium channels (or an adenylyl cyclase/G protein signal transduction mechanism) which may be CNS co-localised and functionally integrated. In addition, the α-2 agonist clonidine (eg. 75-300 μg, thrice daily), the cannabinoid agonist Δ⁹-THC (eg 2.5-5 mg daily)and CCBs may be additive in this respect.

(b) angiotensin-linked calcium channels: angiotensin receptors are coupled to a G-protein effector system involving adenylyl cylclase and phospholipase C. Receptor stimulation leads to calcium/calmodulin-dependent protein kinase activation and increased intracellular calcium resulting in arterial smooth muscle contraction and an increased in vivo blood pressure. This is controlled clinically by oral once daily angiotensin II receptor antagonists in current use, including irbesarten (eg 75-300 mg), losarten (eg 50-100 mg) and candesarten (eg. 4-16 mg). Their anti-hypertensive actions are enhanced by conventional CCBs also used clinically for this purpose.

(c) nucleotide/nucleoside-linked calcium channels: ATP is a sympathetic system neurotransmitter which evokes a receptor-operated calcium current in atrial smooth muscle. Unlike voltage-operated calcium channels, ATP-activated channels respond directly to ligand without the involvement of a diffusible secondary messenger system. Likewise, the latter channels are resistant to conventional calcium channel blocking agents such as nifedipine, Mg²⁺ and Cd²⁺. Thus, ATP-activated channels provide a distinctive mechanism for excitatory synaptic transmission and calcium entry into smooth muscle.

The active ingredients may be administered as a combined dose or as discrete doses. While it may be possible for an active ingredient to be administered alone, it is preferable to present it to a subject as a pharmaceutical composition. Where the CCB is not inherently long-acting, it is administered in a sustained-release formulation. The preparation of sustained-release formulations is well know to the person skilled in the art and is described in references such as Remington's Pharmaceutical Sciences, Chapter 91, pages 1976-93, 18^{th} Edition, MACK Publishing Company. Dosage forms may include oral, parenteral, transdermal forms or implants. It will be understood that a sustained-release dosage form while releasing the active ingredient at a rate such that adverse effects are avoided or minimized, will nevertheless release the active ingredient at a rate which provides the useful clinical effects of the invention.

Oral sustained-release dosage forms may include suitable coatings around particles, tablets, capsules etc or the suspension or distribution of the active agent within a polymeric matrix which slowly releases the active agent.

Parenteral sustained-release dosage forms may include emulsions, solutions and suspensions.

Transdermal sustained-release forms may include ointments, lotions, gels etc in which the active ingredients are suspended as slow release particles or liposomes. Alternatively, patches may be used which may comprise a microporous membrane made from suitable materials such as cellulose nitrate/acetate, propylene and polycarbonates. The patches may also contain a suitable skin adhesive and backing materials.

Implants, eg subcutaneous implants, may comprise a drug-bearing polymeric device wherein the polymer is biocompatible and non-toxic. Suitable polymers may include hydrogels, silcones, polyeythylenes and biodegradable polymers.

The composition containing the active ingredient may contain any suitable carriers, diluents or excipients. These include all conventional solvents, dispersion media, fillers, solid carriers, coatings, antifungal and antibacterial agents, surfactants, isotonic and absorption agents and the like. It will be understood that the compositions of the invention may also include other physiologically-active agents, where appropriate.

The carrier, diluent or excipient must be pharmaceutically “acceptable” in the sense of being compatible with the other ingredients of the composition and not injurous to the subject. Preferably the compositions are suitable for oral administration. The compositions may conveniently be presented in unit dosage form and may be prepared by any methods well known in the art of pharmacy. Such methods include the step of bringing into association the active ingredient with the carrier which constitutes one or more accessory ingredients. In general, the compositions are prepared by uniformly and intimately bringing into association the active ingredient with liquid carriers or finely divided solid carriers or both, and then if necessary shaping the product.

Compositions of the present invention suitable for oral administration may be presented as discrete units such as, capsules, sachets or tablets each containing a pre-determined amount of the active ingredient; as a powder or granules; as a solution or a suspension in an aqueous or non-aqueous liquid; or as an oil-in-water liquid emulsion or a water-in-oil liquid emulsion. The active ingredient may also be presented as a bolus or paste.

A tablet may be made by compression or moulding optionally with one or more accessory ingredients. Compressed tablets may be prepared by compressing in a suitable machine the active ingredient in a free-flowing form such as a powder or granules, optionally mixed with a binder (eg, inert diluent, preservative disintegrant (eg, sodium starch glycolate, cross-linked polyvinyl pyrrolidone, cross-linked sodium carboxymethyl cellulose) surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

Where appropriate, the active ingredients may be administered as a salt or pro-drug thereof.

The term “salt”, or “pro-drug” includes any pharmaceutically acceptable salt, ester, solvate, hydrate or any other compound which, upon administration to the recipient is capable of providing (directly or indirectly) the μORA, NMDA glutamate receptor modulator or CCB. The term “pro-drug” is used in its broadest sense and encompasses those derivatives that are converted in vivo to the active ingredients used in the invention. Such derivatives are well known to those skilled in the art.

Suitable pharmaceutically acceptable salts include, but are not limited to salts of pharmaceutically acceptable inorganic acids such as hydrochloric, sulphuric, phosphoric, nitric, carbonic, boric, sulfamic and hydrobromic acids, or salts of pharmaceutically acceptable organic acids such as acetic, propionic, butyric, tartaric, hydroxymaleic, fumaric, maleic, citric, lactic, mucic, gluconic, benzoic, succinic, oxalic, phenylacetic, methanesulphonic, toluenesulphonic, benezenesulphonic, salicyclic, sulphanilic, aspartic, glutamic, stearic, palmitic, oleic, lauric, pantothenic, tannic, ascorbic, and valeric acids.

Base salts include, but are not limited to, those formed with pharmaceutically acceptable cations, such as sodium, potassium, lithium, calcium, magnesium, ammonium and alkylammonium.

Basic nitrogen-containing groups may be quarternised with such agents as lower alkyl halide, such as methyl, ethyl, propyl and butyl chlorides, bromides and iodides; dialkyl sulfates like dimethyl and diethyl sulfate; and others.

In a preferred form of the invention, the combination of μORA, NMDA glutamate receptor modulator and CCB is administered to the subject for some 12 to 24 weeks. In a further preferred form, following the administration of the combination for about 12 to 24 weeks, should the beneficial effects of naltrexone no longer be maintained, the treatment may be followed by administration of the CCB alone for as long as its safe and beneficial activity persists. This can be determined by the attending physician.

Preferably, the substances of addiction which may be treated by the present invention include alcohol and volatile solvents such as paint solvents. However, it is recognised that the invention may also be useful in treating several substance addictions in the one subject such as opiate/alcohol or alcohol/volatile solvent etc. The invention may also have utility in the treatment of nicotine addiction. In such a case, the combination of the invention may further comprise a ganglion nicotinic receptor antagonist, such as mecamylamine; or a nicotinic cholinergic receptor antagonist, such as bupropion; or γ-vinylGABA (vigabactin) or a κ-opioid agonist.

The subject, depending on the nature of the addiction, may be a suitably detoxified subject, preferably detoxified for at least 5-10 days. The long-term substance-dependent subject is generally symptomatically detoxified prior to receiving the new combination treatments of the invention. Processes of detoxification are well known by the skilled person.

Opiate-dependent subjects treated by the combinations of the present invention are also detoxified. Detoxification methods are known in the art. For opiate addicts, detoxification may involve an acute dose of naltrexone and subsequent symptomatic treatment. About 6-10 days after detoxification, presence of residual withdrawal may be determined by the Narcan test, involving the administration of the opioid receptor antagonist naloxone. The presence or absence of symptoms of residual morphine withdrawal are then observed over a 20-30 minute interval.

Alcoholics may optionally be detoxified. Again, detoxification methods are known within the art. For complete detoxification, alcohol addicts usually receive diazepam and symptomatic treatment for 5-6 days. If the prior symptoms of alcoholism, such as impaired cognition and motor skills are absent, or only minimally present, the subject is considered to be detoxified.

It will be recognised that the μORA, NMDA glutamate receptor modulator and CCB can be administered simultaneously, or sequentially, either one immediately after the other or separated by a suitable interval. Where the aforementioned components are administered simultaneously, they can be administered either as discrete dosage forms or in a combined form, ie, a composition containing two or three of these components.

Thus, in a further aspect, the invention also provides compositions for use in the present invention containing at least two of a μORA, an NMDA glutamate receptor modulator and a CCB which is long lasting or in a sustained-release form or which is nimodipine in rapid release form.

In another aspect, the invention provides a kit for use in the present invention comprising at least two of, preferably all of a μORA, an NMDA glutamate receptor modulator and a CCB which is long-acting or in sustained-release form, or which is nimodipine in rapid release form, wherein said kit is in compartmentalized form adapted for the simultaneous or sequential administration of the at least 2 or all 3 of the μORA, NMDA glutamate receptor modulator and CCB.

In still another aspect, the invention provides for the use of a μORA in the preparation of a medicament for the treatment of substance addiction, wherein said medicament is adapted for administration to a subject in combination with an NMDA glutamate receptor modulator and a CCB, which is long-acting or in sustained-release form, or which is nimodipine in rapid release form.

The invention also provides for the use of a CCB which is long-acting or in sustained-release form, or which is nimodipine in rapid release form, in the preparation of a medicament for the treatment of substance addiction, wherein said medicament is adapted for administration in combination with a μORA and an NMDA glutamate receptor modulator.

Still a further aspect relates to the use of an NMDA glutamate receptor modulator in the preparation of a medicament for the treatment of substance addiction where said medicament is adapted for administration in combination with a CCB, which is long-acting or in sustained-release form, or which is nimodipine in rapid release form, and a μORA.

The invention further provides for the use of a μORA, an NMDA glutamate receptor modulator and a CCB in the preparation of a medicament for the treatment of substance addiction, wherein the CCB is long-acting or formulated in the medicament such that it is in a sustained-release form.

The μORA, NMDA glutamate receptor modulator and CCB are each administered in a treatment-effective amount. A treatment-effective amount is the amount of each active ingredient which, when administered according to the desired dosing regimen, provides the desired therapeutic effect such as remission, cure or elimination of or reduction in craving of, for example at least 30% (more preferably at least 50%, even more preferably at least 70%, 80% or 90%), or prevention or delay in relapse to chronic dependence on the substance of addiction or increase in abstinence for the preferred and other drugs of abuse up to total abstinence. The active ingredients may be administered in additive or synergistically effective amounts. Preferably the active ingredients are administered in combination, one or more of the components being administered in an amount which is less than that which gives the desired therapeutic effect when administered alone, ie one or more of the active ingredients are administered in synergistically-effective amounts. In a preferred form, one or more of each of the active ingredients is administered at about half the conventionally-recommended therapeutic dosage rates. Thus, approximately half therapeutic doses of each component of the combination dosage forms may be formulated such that the combinations contain the required amount of each component or alternatively, may contain a larger amount but the dosage form eg a tablet, is formed such that it can easily be divided without affecting the sustained-release mechanism, eg scoring of a tablet.

Suitable dosage amounts and regimens can be determined by the attending physician and may depend on the particular substance addiction being treated, as well as the general health, age and weight of the subject.

Where disturbing or excessive vasodilatory or cardio-accelerant symptoms are produced by higher dosages of CCBs, they can be diminished or prevented by the administration of 20 mg propranolol (Inderal^{R}) given approximately 20 minutes prior to administration of the CCB.

A preferred embodiment of the invention is one in which the components of the combination may be administered together or individually on a once, twice or thrice daily basis (with suitable adjustment of dosage), although it is preferable to treat patients on a once daily basis to maximize patient compliance.

Examples of Preferred Combinations Include:

(i) 12.5-100 mg of naltrexone, ˜1000-2000 mg of acamprosate and 80-480 mg of sustained-release verapamil or 2.5-10, mg of long-acting amlodipine. Thus for example, the treatment can be based on a once daily dose of 25 or 50 mg naltrexone, a thrice daily dose of 333 mg or 666 mg of acamprosate (or a once or twice daily dose of ˜1000 mg) and a once daily dose of 120-240 mg of sustained-release verapamil or a once daily dose of 2.5-10 mg of long-acting amlodipine.

(ii) 12.5-100 mg of naltrexone, ˜1000-2000 of acamprosate and 2.5-20 mg of sustained-release felodipine or 15-120 mg of sustained-release nifedipine. Thus for example, the treatment can be based on a once daily dose of 25 or 50 mg naltrexone, a thrice daily dose of 333 mg or 666 mg of acamprosate (or a once or twice daily dose of ˜1000 mg) and a once daily dose of 2.5-5 mg of sustained-release felodipine (eg. Plendyl^{R}) or a once daily dose of 30-60 mg of sustained-release nifedipine (eg., Adalat Oros^{R}).

(iii) 12.5-100 mg of naltrexone, ˜1000-2000 mg of acamprosate and 10-40 mg of nitrendipine, 30-60 mg of nicardipine or 20-40 mg of nisoldipine. Thus for example, the treatment can be based on a once daily dose of 25 or 50 mg naltrexone, a thrice daily dose of 333 mg or 666 mg of acamprosate (or a once or twice daily dose of ˜1000 mg) and a once or twice daily dose of 5-20 mg of nitrendipine, a once or twice daily dose of 15-30 mg of nicardpine or a once or twice daily dose of 10-20 mg of nisoldipine.

(iv) 12.5-100 mg of naltrexone, ˜1000-2000 mg of acamprosate and 2-8 mg of long-acting lacidipine, 120-360 mg of sustained-release diltiazem or 15-240 mg of rapid release nimodipine. Thus for example, the treatment can be based on a once daily dose of 25 or 50 mg naltrexone, a thrice daily dose of 333 mg or 666 mg of acamprosate (or a once or twice daily dose of ˜1000 mg) and a once daily dose of 2-8 mg of lacidipine, once daily dose of 120-360 mg of diltiazem or 30-60 mg of nimodipine given three or four time daily.

(v) 12.5-100 mg naltrexone, ˜1000-2000 mg of acamprosate and 2.5-20 mg of sustained-release isradipine or 20-60 mg of flunarizine. Thus for example, the treatement can be based on a once daily dose of 25 or 50 mg naltrexone, a thrice daily dose of 333 mg or 666 mg acamprosate (or a once or twice daily dose of ˜1000 mg), and a once daily dose of 5-10 mg of sustained-release isradipine, or a once, twice or thrice daily dose of 10-20 mg of flunarizine.

Throughout this specification and the claims which follow, unless the context requires otherwise, the word “comprise” and variations such as “comprises” and “comprising” will be understood to imply the inclusion of a stated integer or step or group of integers but not the exclusion of any other integer or step or group of integers. Those skilled in the art will appreciate that the invention described herein is susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and, any and all combinations of anytwo or more of said steps or features.

The invention will now be described with reference to the following Examples which are intended for the purpose of illustration only and should not be construed as limiting the scope of the generality hereinbefore described.

### EXAMPLES

### Example 1

Particularly suitable examples of combinations or compositions of naltrexone/acamprosate/verapamil or amlodipine in accordance with the present invention may include:

(a) Once daily naltrexone (12.5 mg) given together with, a thrice daily dose of 333 mg or 666 mg acamprosate (or a once or twice daily dose of ˜1000 mg) and once daily sustained-release verapamil (80 mg, 90 mg, 120 mg, 160 mg, 180 mg, 240 mg, 320 mg, 360 mg or 480 mg).

(b) Once daily naltrexone (25 mg) given together with a thrice daily dose of 333 mg or 666 mg acamprosate (or a once or twice daily dose of ˜1000 mg) and once daily sustained-release verapamil (80 mg, 90 mg, 120 mg, 160 mg, 180 mg, 240 mg, 320 mg, 360 mg or 480 mg).

(c) Once daily naltrexone (50 mg) given together with a thrice daily dose of 333 mg or 666 mg acamprosate (or a once or twice daily dose of ˜1000 mg) and once daily sustained-release verapamil (80 mg, 90 mg, 120 mg, 160 mg, 180 mg, 240 mg, 320 mg, 360 mg or 480 mg).

(d) Once daily naltrexone (˜1000 mg) given together with a thrice daily dose of 333 mg or 666 mg acamprosate (or a once of twice daily dose of 1000 mg) and once daily sustained-release verapamil (80 mg, 90 mg, 120 mg, 160 mg, 180 mg, 240 mg, 320 mg, 360 mg or 480 mg). A succeeding 100 mg daily dose of naltrexone can be given at second daily intervals.

(e) Once daily naltrexone (12.5 mg) given together with a thrice daily dose of 333 mg or 666 mg acamprosate (or a once or twice daily dose of ˜1000 mg) and once daily long-acting amlodipine (2.5 mg, 5 mg, 10 mg, or 20 mg).

(f) Once daily naltrexone (25 mg) given together with a thrice daily dose of 333 mg or 666 mg acamprosate (or a once or twice daily dose of 1000 mg) and once daily long-acting amlodipine (2.5 mg, 5 mg, 10 mg, or 20 mg).

(g) Once daily naltrexone (50 mg) given together with a thrice daily dose of 333 mg or 666 mg acamprosate (or a once or twice daily dose of ˜1000 mg) and once daily long-acting amlodipine (2.5 mg, 5 mg, 10 mg, or 20 mg).

(h) Once daily naltrexone (100 mg) given together with a thrice daily dose of 333 mg or 666 mg acamprosate (or a once or twice daily dose of 1000 mg) and once daily long-acting amlodipine (2.5 mg, 5 mg, 10 mg, or 20 mg). A succeeding 100 mg dose of naltrexone can be given at second daily intervals.

### Example 2

Examples of combinations or compositons of naltrexone/acamprosate/felodipine or nifedipine in accordance with the present invention may include:

(a) Once daily naltrexone (12.5 mg) given together with a thrice daily dose of 333 mg or 666 mg acamprosate (or a once or twice daily dose of ˜1000 mg) and once daily sustained-release felodipine (2.5 mg, 5 mg, 7.5 mg, 10 mg, 15 mg or 20 mg).

(b) Once daily naltrexone (25 mg) given together with a thrice daily dose of 333 mg or 666 mg acamprosate (or a once or twice daily dose of ˜1000 mg) and once daily sustained release felodipine (2.5 mg, 5 mg, 7.5 mg, 10 mg, 15 mg or 20 mg).

(c) Once daily naltrexone (50 mg) given with a thrice daily dose of 333 mg or 666 mg acamprosate (or a once or twice daily dose of ˜1000 mg) and once daily sustained-release felodipine (2.5 mg, 5 mg, 7.5 mg, 10 mg, 15 mg or 20 mg).

(d) Once daily naltrexone (100 mg) given with a thrice daily dose of 333 mg or 666 mg acamprosate (or a once or twice daily dose of ˜1000 mg) and once daily sustained-release felodipine (2.5 mg, 5 mg, 7.5 mg, 10 mg, 15 mg or 20 mg). A succeeding 100 mg daily dose of naltrexone can be given at second daily intervals.

(e) Once daily naltrexone (12.5 mg) given with a thrice daily dose of 333 mg or 666 mg acamprosate (or a once or twice daily dose of 1000 mg) and once daily sustained-release nifedipine (15 mg, 30 mg, 45 mg, 60 mg, 90 mg or 120 mg).

(f) Once daily naltrexone (25 mg) given together with a thrice daily dose of 333 mg or 666 mg acamprosate (or a once or twice daily dose of ˜1000 mg) and once daily sustained-release nifedipine (15 mg, 30 mg, 45 mg, 60 mg, 90 mg or 120 mg).

(g) Once daily naltrexone (50 mg) given with a thrice daily dose of 333 mg or 666 mg acamprosate (or a once or twice daily dose of ˜1000 mg) and once daily sustained-release nifedipine (15 mg, 30 mg, 45 mg, 60 mg, 90 mg or 120 mg).

(h) Once daily naltrexone (100 mg) given with a thrice daily dose of 333 mg or 666 mg acamprosate (or a once or twice daily dose of ˜1000 mg) and once daily sustained-release nifedipine (15 mg, 30 mg, 45 mg, 60 mg, 90 mg or 120 mg). A succeeding 100 mg dose of naltrexone can be given at second daily intervals.

### Example 3

Examples of combinations or compositions of naltrexone/acamprosate/nitrendipine or nicardipine or nisoldipine in accordance with the present invention may include:

(a) daily 12.5 mg naltrexone and a thrice daily dose of 333 mg or 666 mg acamprosate (or a once or twice daily dose of ˜1000 mg) and 5 mg, 10 mg, 15 mg, or 20 mg of long-acting nitrendipine once or twice daily.

(b) daily 25 mg naltrexone and a thrice daily dose of 333 mg or 666 mg acamprosate. (or a once or twice daily dose of ˜1000 mg) and 5 mg, 10 mg, 15 mg, or 20 mg of long-acting nitrendipine once or twice daily.

(c) daily 50 mg naltrexone and a thrice daily dose of 333 mg or 666 mg acamprosate (or a once or twice daily dose of 1000 mg) and 5 mg, 10 mg, 15 mg, or 20 mg of long-acting nitrendipine once or twice daily.

(d) daily 100 mg naltrexone and a thrice daily dose of 333 mg or 666 mg acamprosate. (or a once or twice daily dose of ˜1000 mg) and 5 mg, 10 mg, 15 mg, or 20 mg of long-acting nitrendipine once or twice daily.

(e) daily 12.5 mg naltrexone and a thrice daily dose of 333 mg or 666 mg acamprosate (or a once or twice daily dose of ˜1000 mg) and 15 mg, 30 mg, 45 mg, or 60 mg of sustained-release nicardipine as a once or twice daily.

(f) daily 25 mg naltrexone and a thrice daily dose of 333 mg or 666 mg acamprosate (or a once or twice daily dose of ˜1000 mg) and 15 mg, 30 mg, 45 mg, or 60 mg of sustained-release nicardipine once or twice daily.

(g) daily 50 mg naltrexone and a thrice daily dose of 333 mg or 666 mg acamprosate (or a once or twice daily dose of ˜1000 mg) and 15 mg, 30 mg, 45 mg, or 60 mg of sustained-release nicardipine once or twice daily.

(h) daily 100 mg naltrexone and a thrice daily dose of 333 mg or 666 mg acamprosate (or a once or twice daily dose of ˜1000 mg) and 15 mg, 30 mg, 45 mg, or 60 mg of sustained-release nicardipine once or twice daily.

(i) daily 12.5 mg naltrexone and a thrice daily dose of 333 mg or 666 mg acamprosate, (or a once or twice daily dose of ˜1000 mg) and 10 mg, 20 mg, 30 mg, or 40 mg of sustained-release nisoldipine once or twice daily.

(j) daily 25 mg naltrexone and a thrice daily dose of 333 mg or 666 mg acamprosate (or a once or twice daily dose of ˜1000 mg) and 10 mg, 20 mg, 30 mg, or 40 mg of sustained-release nisoldipine once or twice daily.

(k) daily 50 mg naltrexone and a thrice daily dose of 333 mg or 666 mg acamprosate (or a once or twice daily dose of ˜1000 mg) and 10 mg, 20 mg, 30 mg, or 40 mg of sustained-release nisoldipine once or twice daily.

(l) daily 100 mg naltrexone and a thrice daily dose of 333 mg or 666 mg acamprosate (or a once or twice daily dose of ˜1000 mg) and 10 mg, 20 mg, 30 mg, or 40 mg of sustained-release nisoldipine once or twice daily.

### Example 4

Examples of combinations or compositions of naltrexone/acamprosate/lacidipine or diltiazem or nimodipine in accordance with the present invention include:

(a) daily 12.5 mg naltrexone and a thrice daily dose of 333 mg or 666 mg acamprosate (or a once or twice daily dose of ˜1000 mg) and 2 mg, 4 mg, 6 mg or 8 mg of long-acting lacidipine once daily.

(b) daily 25 mg naltrexone and a thrice daily dose of 333 mg or 666 mg acamprosate. (or a once or twice daily dose of ˜1000 mg) and 2 mg, 4 mg, 6 mg or 8 mg of long-acting lacidipine once daily.

(c) daily 50 mg naltrexone and a thrice daily dose of 333 mg or 666 mg acamprosate (or a once or twice daily dose of ˜1000 mg) and 2 mg, 4 mg, 6 mg or 8 mg of long-acting lacidipine once daily.

(d) daily 100 mg naltrexone and a thrice daily dose of 333 mg or 666 mg acamprosate (or a once or twice daily dose of ˜1000 mg) and 2 mg, 4 mg, 6 mg or 8 mg of long-acting lacidipine once daily.

(e) daily 12.5 mg naltrexone and a thrice daily dose of 333 mg or 666 mg acamprosate (or a once or twice daily dose of ˜1000 mg) and 90 mg, 180 mg, 240 mg or 360 mg of sustained-release diltiazem once daily.

(f) daily 25 mg naltrexone and a thrice daily dose of 333 mg or 666 mg acamprosate (or a once or twice daily dose of ˜1000 mg) and 90 mg, 180 mg, 240 mg or 360 mg of sustained-release diltiazem once daily.

(g) daily 50 mg naltrexone and a thrice daily dose of 333 mg or 666 mg acamprosate (or a once or twice daily dose of ˜1000 mg) and 90 mg, 180 mg, 240 mg or 360 mg of sustained-release diltiazem once daily.

h) daily 100 mg naltrexone and a thrice daily dose of 333 mg or 666 mg acamprosate (or a once or twice daily dose of ˜1000 mg) and 90 mg, 180 mg, 240 mg or 360 mg of sustained-release diltiazem once daily.

(i) daily 12.5 mg naltrexone and a thrice daily dose of 333 mg or 666 mg acamprosate (or a once or twice daily dose of ˜1000 mg) and 15 mg, 30 mg, 45 mg or 60 mg of nimodipine three or four times daily.

(j) daily 25 mg naltrexone and a thrice daily dose of 333 mg or 666 mg acamprosate (or a once or twice daily dose of ˜1000 mg) and 15 mg, 30 mg, 45 mg or 60 mg of nimodipine three or four times daily.

(k) daily 50 mg naltrexone and a thrice daily dose of 333 mg or 666 mg acamprosate (or a once or twice daily dose of ˜1000 mg) and 15 mg, 30 mg, 45 mg or 60 mg of nimodipine three or four times daily.

(l) daily 100 mg naltrexone and a thrice daily dose of 333 mg or 666 mg acamprosate (or a once or twice daily dose of ˜1000 mg) and 15 mg, 30 mg, 45 mg or 60 mg of nimodipine three or four times daily.

### Example 5

Examples of combinations or compositions of naltrexone/acamprosate/isradipine or flunarizine in accordance with the present invention include:

(a) daily 12.5 mg naltrexone and a thrice daily dose of 333 mg or 666 mg acamprosate (or a once or twice daily dose of ˜1000 mg) and 2.5 mg, 5 mg, 10 mg or 20 mg of sustained-release isradipine once daily.

(b) daily 25 mg naltrexone and a thrice daily dose of 333 mg or 666 mg acamprosate (or a once or twice daily dose of ˜1000 mg) and 2.5 mg, 5 mg, 10 mg or 20 mg of sustained-release isradipine once daily.

(c) daily 50 mg naltrexone and a thrice daily dose of 333 mg or 666 mg acamprosate (or a once or twice daily dose of ˜1000 mg) and 2.5 mg, 5 mg, 10 mg or 20 mg of sustained-release isradipine once daily.

(d) daily 100 mg naltrexone and a thrice daily dose of 333 mg or 666 mg acamprosate (or a once or twice daily dose of ˜1000 mg) and 2.5 mg, 5 mg, 10 mg or 20 mg of sustained-release isradipine-once daily.

(e) daily 12.5 mg naltrexone and a thrice daily dose of 333 mg or 666 mg acamprosate (or a once or twice daily dose of ˜1000 mg) and 10 mg or 20 mg of flunarizine as a once, twice or thrice daily dose.

(f) daily 25 mg naltrexone and a thrice daily dose of 333 mg or 666 mg acamprosate (or a once or twice daily dose of ˜1000 mg) and 10 mg or 20 mg of flunarizine as a once, twice or thrice daily dose.

(g) daily 50 mg naltrexone and a thrice daily dose of 333 mg or 666 mg acamprosate (or a once or twice daily dose of ˜1000 mg) and 10 mg or 20 mg of flunarizine as a once, twice or thrice daily dose.

(h) daily 100 mg naltrexone and a thrice daily dose of 333 mg or 666 mg acamprosate (or a once or twice daily dose of ˜1000 mg) and 10 mg or 20 mg of flunarizine as a once, twice or thrice daily dose.

### Example 6

6 Detoxified alcoholic patients were treated in an N-of-1Double Blind Placebo-Controlled Cross-Over Clinical Trial with a daily dose of naltrexone (25 mg), acamprosate (˜1 g) and one of amlodipine, felodipine or verapamil (at its specified or half specified therapeutic dose).

The patient characteristics, their randomised active and control treatments and their outcomes, and the key and ancillary parameters on which the progressive comparative weekly safeties and efficacies of the active and control treatments were determined for each of the 6 double-blind trial subjects are all shown in Table 1. The following information is provided to facilitate the interpretation of these data:

(i) Each trial subject received sequentially 3 different but operationally-related individual treatments, each on an oral once daily basis, labelled, for convenience, A (which may randomly be an active or control treatment), B (wash-out) and C (which may also randomly be a control or wash-out treatment)

(ii) To permit their optimal comparison by the same test subject, treatments A & C were both given for the same and longest length of time available before the trial was terminated. Patient and clinician bias in interpretation was minimised or excluded by giving these treatments on a randomised double-blind basis which ensured that neither knew if the active or control treatment was given first or last. Thus sometimes, labelled treatment A or C was the active or control treatment and vice versa. The actual drug components of treatments A, B and C for each trial subject, and the duration of each treatment are also shown in Table 1 in each case.

(iii) Administration of active and control treatments was always separated by a daily wash-out treatment (B), given for one week, to ensure that there was no carry-over effect from the previous treatment.

(iv) One unit of alcohol was taken as 425 ml light beer/lager or 285 ml full strength beer or 100 ml wine or 30 ml spirits.

(v) Scoring of key parameters was based on questionaries completed by the subject. For the audit score, an initial base-line questionnaire preceding treatment and subsequently, a similar weekly questionnaire, following each week of treatment was conducted by the trial clinician and trial subject. The subject's answers were assigned a value and the score for all 10 questions totalled (out of 40). A total of 13+ is indicated as alcohol dependent, 8+ as a hazardous drinker and less than 8 a safe drinker, with 0 indicating abstinence. Levels of Craving, tendency to Relapse and degree of Abstinence were rated by the subject on a scale of 0-10, with 0,0 indicating absence of craving and relapse respectively, and 10 indicating total abstinence from alcohol and volatile inhalant.

In two subjects, the administered dose of amlodipine (5 mg) produced a number of initial transient side-effects which were generally prevented by the prior 20-30 minute administration of 20 mg propranolol which generally had no effect on blood pressure.

TABLE 1
Double-Blind Placebo-controlled Crossover Therapeutic Trial in detoxified Alcohol subjects.

| | | | | Date & Treatment* | | | Key Parameters | | | | | Improved | | | |
|---|---|---|---|---|---|---|---|---|---|---|---|---|---|---|---|
| | | | Primary Drug Use | (mg/d) | | | Audit | | Re- | Ab- | | (1) Ancillary Parameters | | | |
| No | M/F | Age | H/M | A | B | C | Score | Craving | lapse | stinence | Side-Effects | Energy | Thinking | In-control | Patient's View |
| 1 | F | 47 | Alc-6-10 Units/d 10 Y | Start: 17/10/00 | | | 30 | 9 | 2 | 0 | | | | | |
| | | | | N25 + A333 × 3 + Pl | N25 + A333 × 3 | N25 + A333 × 3 + Aml5 | | | | | | | | | |
| | | | | 24/10/00 | | | 18 | 0 | 0 | 10 | Minimal | 1+ | 1+ | 1+ | Progress+ |
| | | | | 31/10/00 | | | 17 | 0 | 0 | 10 | Nil | 1+ | 1+ | 1+ | Progress+ |
| | | | | 06/11/00 | | | 14 | 0 | 0 | 10 | Minimal | 1+ | 1+ | 1+ | Progress+ |
| | | | | 14/11/00 | | | 14 | 0 | 0 | 10 | Nil | 1+ | 1+ | 1+ | Progress+ |
| | | | | 21/11/00 | | | 14 | 0 | 0 | 10 | Nil | 1+ | 1+ | 1+ | Progress+ |
| | | | | | 28/11/00 | | 14 | 0 | 0 | 10 | Nil | 1+ | 1+ | 1+ | Progress+ |
| | | | | | | 05/12/00 | 12 | 0 | 0 | 10 | Nil | 1+ | 1+ | 1+ | Progress+ |
| | | | | | | 12/12/00 | 11 | 0 | 0 | 10 | Mod 2/7 | 1+ | 1+ | 1+ | Progress+ |
| | | | | | | 19/12/00 | 13 | 0 | 0 | 10 | Mild | 1+ | 1+ | 1+ | Progress+ |
| | | | | | | 26/12/00 | 13 | 0 | 0 | 10 | Nil | 1++ | 1++ | 1++ | Progress++ |
| | | | | | | 03/01/01 | 5 | 0 | 0 | 10 | Nil | 1+++ | 1+++ | 1+++ | Progress+++ |
| 2 | F | 50 | Alc 6-10 Units/d 8 Y | Start: 12/10/00 | | | 23 | 8 | 8 | 0 | | | | | |
| | | | | N25 + A333 × 3 + Fel2.5 | N25 + A333 × 3 | N25 + A333 × 3 + Pl | | | | | | | | | |
| | | | | 19/10/00 | | | 20 | 4 | 2 | 8 | Mod | 0 | 0 | 0 | Progress 0 |
| | | | | 26/10/00 | | | 17 | 0 | 0 | 9 | Mod | 1+/− | 1+/− | 1+/− | Progress+/− |
| | | | | 08/11/00 | | | 15 | 0 | 0 | 10 | Mild | 1+ | 1+ | 1+ | Progress+ |
| | | | | 16/11/00 | | | 15 | 0 | 0 | 10 | Mild | 1+ | 1+ | 1+ | Progress+ |
| | | | | 23/11/00 | | | 15 | 0 | 0 | 10 | Mild | 1++ | 1++ | 1++ | Progress++ |
| | | | | | 30/11/00 | | 15 | 2 | 1 | 10 | Nil | 1+ | 1+ | 1+ | Progress+ |
| | | | | | | 07/12/00 | 17 | 2 | 2 | 8 | Nil | 1+ | 1+ | 1+ | Progress+ |
| | | | | | | 14/12/00 | 17 | 3 | 6 | 8 | Nil | 1+/− | 1+/− | 1+/− | Progress+/− |
| | | | | | | 22/12/00 | 18 | 4 | 7 | 4 | Nil | 1+/− | 1+/− | 1+/− | Progress+/− |
| | | | | | | 30/12/00 | 18 | 2 | 2 | 10 | Nil | 1+ | 1+ | 1+ | Progress+ |
| | | | | | | 08/01/01 | 17 | 0 | 0 | 10 | Nil | 1++ | 1++ | 1++ | Progress++ |
| 3 | F | 51 | Alc 10-15 Units/d | Start: 07/11/00 | | | 29 | 8 | 5 | 5 | | | | | |
| | | | | N25 + A333 × 3 + Fel2.5 | N25 + A333 × 3 | N25 + A333 × 3 + Pl | | | | | | | | | |
| | | | | 14/11/00 | | | 31 | 6 | 5 | 8 | Nil | 1+/− | 1+/− | 1+/− | Progress+/− |
| | | | | 21/11/00 | | | 29 | 6 | 5 | 8 | Mild | 1+ | 1+ | 1++ | Progress+ |
| | | | | 28/11/00 | | | 29 | 5 | 5 | 10 | Mild | 1++ | 1++ | 1++ | Progress++ |
| | | | | 05/12/00 | | | 29 | 5 | 2 | 10 | Mild | 1+++ | 1+++ | 1+++ | Progress+++ |
| | | | | | 12/12/00 | | 29 | 7 | 3 | 8 | Nil | 1++ | 1++ | 1++ | Progress++ |
| | | | | | | 19/12/00 | 31 | 7 | 8 | 1 | Nil | 1+ | 1+ | 1+ | Progress+ |
| | | | | | | 27/12/00 | 31 | 9 | 10 | 1 | Nil | 1+/− | 1+/− | 1+/− | Progress+/− |
| | | | | | | 04/01/01 | 30 | 9 | 10 | 1 | Nil | 1+/− | 1+/− | 1+/− | Progress+/− |
| | | | | | | 09/01/01 | 30 | 8 | 9 | 1 | Nil | 0 | 0 | 0 | Progress 0 |
| 4 | M | 30 | Alc 20 Units/d | Start: 16/10/00 | | | 12 | 8 | 6 | 6 | | | | | |
| | | | | N25 + A333 × 3 + Pl | N25 + A333 × 3 | N25 + A333 × 3 + Fel2.5 | | | | | | | | | |
| | | | | 23/10/00 | | | 12 | 3 | 3 | 3 | Nil | 1+/− | 1+/− | 0 | Progress+ |
| | | | | 31/10/11 | | | 9 | 3 | 3 | 3 | Nil | 1+ | 1++ | 0 | Progress+ |
| | | | | 06/11/00 | | | 9 | 3 | 3 | 3 | Mild | 1+ | 1++ | 1+ | Progress+ |
| | | | | 13/11/00 | | | 9 | 3 | 3 | 3 | Mild | 1++ | 1++ | 1++ | Progress+ |
| | | | | 20/11/00 | | | 9 | 3 | 3 | 3 | Mild | 1++ | 1++ | 1+++ | Progress+ |
| | | | | | 27/11/00 | | 9 | 3 | 3 | 3 | Mild | 1++ | 1++ | 1++ | Progress+ |
| | | | | | | 03/12/00 | 9 | 3 | 3 | 3 | Nil | 1++ | 1++ | 1++ | Progress+ |
| | | | | | | 10/12/00 | 8 | 2 | 2 | 7 | Nil | 1++ | 1++ | 1++ | Progress++ |
| | | | | | | 17/12/00 | 8 | 2 | 2 | 7 | Nil | 1++ | 1++ | 1++ | Progress++ |
| | | | | | | 24/12/00 | 9 | 2 | 2 | 7 | Nil | 1++ | 1++ | 1++ | Progress++ |
| | | | | | | 03/01/01 | 9 | 2 | 2 | 7 | Nil | 1++ | 1++ | 1++ | Progress++ |
| 5 | M | 26 | H-05-1 g/d | Start: 17/11/00 | | | 17 | 6 | 7 | 3 | | | | | |
| | | | Alc 6-8 Units/d | N25 + A333 × 3 + V180 | N25 + A333 × 3 | N25 + A333 × 3 + Pl | | | | | | | | | |
| | | | | 23/11/00 | | | 7 | 5 | 0 | 10 | Nil | 1+ | 1+ | 1+ | Progress+ |
| | | | | 30/11/00 | | | 6 | 3 | 0 | 10 | Nil | 1+ | 1+ | 1+ | Progress+ |
| | | | | 07/12/00 | | | 4 | 3 | 0 | 10 | Nil | 1+ | 1+ | 1++ | Progress++ |
| | | | | 14/12/00 | | | 4 | 2 | 0 | 10 | Nil | 1++ | 1++ | 1++ | Progress++ |
| | | | | | 21/12/00 | | 8 | 2 | 0 | 10 | Nil | 1++ | 1++ | 1++ | Progress++ |
| | | | | | | 29/12/00 | 8 | 2 | 0 | 9 | Nil | 1++ | 1++ | 1++ | Progress++ |
| | | | | | | 05/01/01 | 9 | 4 | 1 | 7 | Nil | 1+ | 1+ | 1+ | Progress+ |
| | | | | | | 09/01/01 | 9 | 4 | 2 | 6 | Nil | 1+ | 1+ | 1+ | Progress+ |
| 6 | M | 17 | Alc 10-20 Units/d | Start: 10/11/00 | | | 32 | 10 | 10 | 0 | | | | | |
| | | | | N25 + A333 × 3 + Aml5 | N25 + A333 × 3 | N25 + A333 × 3 + Pl | | | | | | | | | |
| | | | | 16/11/00 | | | 28 | 7 | 6 | 6 | Mild | 1+ | 1+ | 1+ | Progress+ |
| | | | | 23/11/00 | | | 26 | 3 | 2 | 10 | Nil | 1++ | 1++ | 1+++ | Progress+++ |
| | | | | 30/11/00 | | | 21 | 3 | 2 | 10 | Nil | 1++ | 1++ | 1+++ | Progress+++ |
| | | | | 06/12/00 | | | 21 | 3 | 0 | 10 | Nil | 1++ | 1++ | 1+++ | Progress+++ |
| | | | | | 14/12/00 | | 23 | 4 | 3 | 8 | Nil | 1+ | 1+ | 1++ | Progress++ |
| | | | | | | 21/12/00 | 23 | 5 | 2 | 7 | Nil | 1+ | 1+ | 1+ | Progress+ |
| | | | | | | 28/12/00 | 22 | 5 | 2 | 6 | Nil | 1+ | 1+ | 1+ | Progress+ |
| | | | | | | 04/01/01 | 26 | 7 | 3 | 7 | Nil | 1+ | 1+ | 1+ | Progress+ |
| | | | | | | 09/01/01 | 26 | 7 | 3 | 7 | Nil | 1+/− | 1+/− | 1+/− | Progress+/− |

**N= Naltrexone
A= Acamprosate
Pl= Placebo
Aml= Amlodipine
Fel= Felodipine
V= Verapamil
Numbers behind each agent indicate the dosage administered.

Table IA relating to open trial detoxified opiate subjects is provided for comparative purposes. Patients addicted to opiates only respond to a treatment regimen of a μORA (naltrexone) and a CCB (without acamproste). However, many opiate (or other substance) addicts also have a secondary addiction to alcohol or use alcohol excessively when they are unable to obtain the opiate of addiction. The present invention can also be used to treat such multi-substance addicted subjects.

TABLE 1A
Double-Blind Placebo-Controlled Cross-over Trial in Detoxified Opiate Subjects

| | | | | | | | Key Parameters | | | | | | | | |
|---|---|---|---|---|---|---|---|---|---|---|---|---|---|---|---|
| | | | Primary Drug Use | Date & Treatment (mg/d) | | | Audit | | | Ab- | | Improved (1)Ancillary Parameters | | | |
| No | M/F | Age | H/M | A | B | C | Score | Craving | Relapse | stinence | Side-Effects | Energy | Thinking | In-control | Patient'View |
| 1 | M | 47 | H-05 g/d 25 Y | Start: 10/10/00 | | | | | | | | | | | |
| | | | M-75 Ml/d 3 Y | N25 + PL | N25 | N25 + V180 | | | | | | | | | |
| | | | | 03/11/00 | | | | 3 | 3 | 8 | Nil | 1+/− | 1+/− | 1+/− | Progress +/− |
| | | | | 15/11/00 | | | | 2 | 2 | 8 | Nil | 1+ | 1+ | 1+ | Progress + |
| | | | | 23/11/00 | | | | 2 | 2 | 3 | Nil | 1+ | 1+ | 1+ | Progress + |
| | | | | 29/11/00 | | | | 0 | 0 | 10 | Nil | 1+ | 1+ | 1+ | Progress + |
| | | | | | 07/12/00 | | | 0 | 0 | 10 | Nil | 1+ | 1+ | 1+ | Progress + |
| | | | | | | 14/12/00 | | 0 | 0 | 10 | Nil | 1++ | 1++ | 1++ | Progress ++ |
| | | | | | | 21/12/00 | | 0 | 0 | 10 | Nil | 1++ | 1++ | 1++ | Progress ++ |
| | | | | | | 28/12/00 | | 0 | 0 | 10 | Nil | 1++ | 1++ | 1++ | Progress ++ |
| | | | | | | 03/01/01 | | 0 | 0 | 10 | Nil | 1++ | 1++ | 1++ | Progress ++ |
| 2 | F | 31 | H-05 g/d 8 Y | Start: 11/11/00 | | | | | | | | | | | |
| | | | M-60-120 Ml/d 6/12 | N25 + Aml 2.5 | N25 | N25 + Pl | | | | | | | | | |
| | | | | 24/11/00 | | | | 0 | 0 | 10 | Mild | 1+ | 1+ | 1+ | Progress + |
| | | | | 01/12/00 | | | | 0 | 0 | 10 | Nil | 1++ | 1++ | 1++ | Progress ++ |
| | | | | 05/12/00 | | | | 0 | 0 | 10 | Nil | 1++ | 1++ | 1++ | Progress ++ |
| | | | | 12/12/00 | | | | 0 | 0 | 10 | Nil | 1+++ | 1+++ | 1+++ | Progress +++ |
| | | | | | 19/12/00 | | | 0 | 0 | 10 | Nil | 1++ | 1++ | 1++ | Progress ++ |
| | | | | | | 26/12/00 | | 0 | 0 | 10 | Nil | 1++ | 1++ | 1++ | Progress ++ |
| | | | | No treatment 1/52 owing to requirements of minor surgery | | | | | | | | | | | |
| | | | | | | 05/01/01 | | 0 | 0 | 10 | Nil | 1+ | 1+ | 1+ | Progress + |
| | | | | | | 09/01/01 | | 2 | 0 | 10 | Nil | 1+ | 1+ | 1+ | Progress + |
| 3 | F | 18 | H-0.5 g/d 2-3 Y | Start: 27/10/00 | | | | | | | | | | | |
| | | | Inhalants 5-8 cans/d | N25 + Fel2.5 | N25 | N5 + Pl | | | | | | | | | |
| | | | | 03/11/00 | | | | 6 | 0 | 9 | Mild | 0 | 0 | 0 | Progress 0 |
| | | | | 10/11/00 | | | | 5 | 0 | 10 | Nil | 1+/− | 1+/− | 1+ | Progress + |
| | | | | 17/11/00 | | | | 3 | 0 | 10 | Nil | 1+ | 1++ | 1++ | Progress ++ |
| | | | | 24/11/00 | | | | 1 | 0 | 10 | Nil | 1+++ | 1+++ | 1+++ | Progress +++ |
| | | | | | 01/12/00 | | | 0 | 0 | 10 | Nil | 1++ | 1+++ | 1+++ | Progress +++ |
| | | | | | | 08/12/00 | | 0 | 0 | 10 | Nil | 1++ | 1++ | 1++ | Progress ++ |
| | | | | | | 15/12/00 | | 2 | 0 | 10 | Nil | 1++ | 1++ | 1++ | Progress ++ |
| | | | | | | 22/12/00 | | 2 | 0 | 10 | Nil | 1++ | 1++ | 1++ | Progress ++ |
| | | | | | | 29/12/00 | | 2 | 0 | 10 | Nil | 1++ | 1++ | 1++ | Progress ++ |
| | | | | | | 05/01/01 | | 2 | 0 | 10 | Nil | 1++ | 1++ | 1++ | Progress ++ |
| 4 | M | 30 | H-1-2 g/d | Start: 16/10/00 | | | | | | | | | | | |
| | | | Alc 20 Units/d | N25 + A333 × 3 + Pl | N25 + A333 × 3 | N25 + A333 × 3 + Fel2.5 | | | | | | | | | |
| | | | | 23/10/00 | | | | 3 | 2 | 6 | Nil | 1+/− | 1+/− | 0 | Progress + |
| | | | | 31/10/00 | | | | 1 | 1 | 8 | Nil | 1+ | 1++ | 1++ | Progress ++ |
| | | | | 05/11/00 | | | | 0 | 0 | 10 | Mild | 1+ | 1++ | 1++ | Progress ++ |
| | | | | 13/11/00 | | | | 0 | 0 | 10 | Mild | 1++ | 1++ | 1++ | Progress ++ |
| | | | | 20/11/00 | | | | 0 | 0 | 10 | Mild | 1++ | 1++ | 1++ | Progress ++ |
| | | | | | 27/11/00 | | | 0 | 0 | 10 | Mild | 1++ | 1++ | 1++ | Progress ++ |
| | | | | | | 03/12/00 | | 0 | 0 | 10 | Nil | 1++ | 1++ | 1+++ | Progress +++ |
| | | | | | | 10/12/00 | | 0 | 0 | 10 | Nil | 1++ | 1++ | 1+++ | Progress +++ |
| | | | | | | 17/12/00 | | 0 | 0 | 10 | Nil | 1++ | 1++ | 1+++ | Progress +++ |
| | | | | | | 24/12/00 | | 0 | 0 | 10 | Nil | 1++ | 1++ | 1+++ | Progress +++ |
| | | | | | | 03/01/01 | | 0 | 0 | 10 | Nil | 1++ | 1++ | 1+++ | Progress +++ |
| 5 | F | 34 | H-1 g/d 8-10 Y | Start: 12/11/00 | | | | 10 | 10 | 0 | | | | | |
| | | | | N25 + Aml2.5 | N25 | N25 + Pl | | | | | | | | | |
| | | | | 18/11/00 | | | | 7 | 0 | 10 | NIL | 1+ | 1+ | 1++ | Progress++ |
| | | | | 24/11/00 | | | | 5 | 0 | 10 | NIL | 1+ | 1++ | 1+++ | Progress+++ |
| | | | | 01/12/00 | | | | 4 | 0 | 10 | NIL | 1++ | 1++ | 1+++ | Progress+++ |
| | | | | 09/12/00 | | | | 3 | 0 | 10 | NIL | 1++ | 1++ | 1+++ | Progress+++ |
| | | | | | 17/12/00 | | | 3 | 0 | 10 | NIL | 1++ | 1++ | 1+++ | Progress+++ |
| | | | | | | 24/12/00 | | 7 | 0 | 10 | NIL | 1+ | 1+ | 1++ | Progress++ |
| | | | | | | 31/12/00 | | 8 | 2 | 7 | NIL | 1+ | 1+ | 1+ | Progress+ |
| | | | | | | 05/01/01 | | 8 | 4 | 5 | NIL | 1+ | 0 | 0 | Progress 0 |
| | | | | | | 05/01/01 | | 8 | 4 | 5 | NIL | 1+ | 0 | 0 | Progress 0 |
| 6 | M | 36 | H-1 g/d 7-8 Y | Start: 12/11/00 | | | | 9 | 3 | 0 | | | | | |
| | | | | N25 + Pl | N25 | N25 + Aml2.5 | | | | | | | | | |
| | | | | 18/11/00 | | | | 8 | 8 | 2 | NIL | 0 | 0 | 0 | Progress 0 |
| | | | | 24/11/00 | | | | 8 | 7 | 3 | NIL | 1+ | 1+ | 1+ | Progress+ |
| | | | | 01/12/00 | | | | 7 | 5 | 6 | NIL | 0 | 0 | 1+ | Progress+ |
| | | | | 09/12/00 | | | | 7 | 2 | 8 | NIL | 0 | 0 | 1+ | Progress+ |
| | | | | | 17/12/00 | | | 7 | 2 | 8 | NIL | 0 | 0 | 1+ | Progress+ |
| | | | | | | 23/12/00 | | 6 | 0 | 10 | NIL | 0 | 0 | 1++ | Progress++ |
| | | | | | | 31/12/00 | | 4 | 0 | 10 | NIL | 1++ | 1++ | 1+++ | Progress+++ |
| | | | | | | 04/01/01 | | 3 | 0 | 10 | NIL | 1++ | 1++ | 1+++ | Progress+++ |
| | | | | | | 09/01/01 | | 2 | 0 | 10 | NIL | 1++ | 1++ | 1+++ | Progress+++ |
| 7 | M | 47 | H-05 g/d 25 Y | Start:14/11/00 | | | | 5 | 3 | 4 | | | | | |
| | | | M-75 mg/d 3 Y | NAll25 + Pl | N25 | N25 + V180 | | | | | | | | | |
| | | | | 03/11/00 | | | | 3 | 3 | 8 | NIL | 1+/− | 1+/− | 1+/− | Progress+/− |
| | | | Alc-2-6 Units/d 8 Y | 15/11/00 | | | | 2 | 2 | 8 | NIL | 1+ | 1+ | 1+ | Progress+ |
| | | | | 23/11/00 | | | | 2 | 2 | 3 | NIL | 1+ | 1+ | 1+ | Progress+ |
| | | | | 29/11/00 | | | | 0 | 0 | 10 | NIL | 1+ | 1+ | 1+ | Progress+ |
| | | | | | 7/12/00 | | | 0 | 0 | 10 | NIL | 1+ | 1+ | 1+ | Progress+ |
| | | | | | | 14/12/00 | | 0 | 0 | 10 | NIL | 1++ | 1++ | 1++ | Progress++ |
| | | | | | | 21/12/00 | | 0 | 0 | 10 | NIL | 1++ | 1++ | 1++ | Progress++ |
| | | | | | | 29/12/00 | | 0 | 0 | 10 | NIL | 1++ | 1++ | 1++ | Progress++ |
| | | | | | | 03/01/01 | | 0 | 0 | 10 | NIL | 1++ | 1++ | 1++ | Progress++ |
| 8 | F | 31 | H-05 g/d 8 Y | Start: 10/10/00 | | | | 5 | 0 | 10 | | | | | |
| | | | M-60-120 Ml/d 6/12 | Nall25 + Aml2.5 | N25 | N25 + Pl | | | | | | | | | |
| | | | | 24/11/00 | | | | 0 | 0 | 10 | Mild | 1+ | 1+ | 1+ | Progress+ |
| | | | Alc6-10 Units/d 2 Y | 01/12/00 | | | | 0 | 0 | 10 | NIL | 1++ | 1++ | 1++ | Progress++ |
| | | | BZ 2 Y | 05/12/00 | | | | 0 | 0 | 10 | NIL | 1++ | 1++ | 1++ | Progress++ |
| | | | | 12/12/00 | | | | 0 | 0 | 10 | NIL | 1+++ | 1+++ | 1+++ | Progress+++ |
| | | | | | 19/12/00 | | | 0 | 0 | 10 | NIL | 1++ | 1++ | 1++ | Progress++ |
| | | | | | | 26/12/00 | | 0 | 0 | 10 | NIL | 1++ | 1++ | 1++ | Progress+ |
| | | | | No treatment 1/52 owing to requirements of minor surgery | | | | | | | | | | | |
| | | | | | | 05/01/00 | | 0 | 0 | 10 | NIL | 1+ | 1+ | 1+ | Progress+ |

### Example 7

Two patients were treated in an open trial with a daily dose of naltrexone, acamprosate and verapamil. Key parameters were evaluated as per Example 6. The results are presented in Table 2, depicting treatment duration, patient characteristics, key parameters and improved parameters and side-effects.

The data for subject 2 suggests that the treatment may also be effective in ameliorating craving and relapse and prolonging abstinence for volatile paint solvent inhalants.

TABLE 2
Open Alc/Inhalant Therapeutic (N + A + CCB) Drug Dependence Trial

| | | | | Date & Treatment* | | |
|---|---|---|---|---|---|---|
| | | | Primary Drug Use | (mg/d) | | |
| No | M/F | Age | (H/M) | A | B | C |
| 1 | F | 51 | Alc 20+ Units/d | Start: July 2000 | | |
| | | | | A666 × 3 | | |
| | | | | 01/07/00 | | |
| | | | | 09/07/00 | | |
| | | | | N50 + A666 × 3 + V240 | | |
| | | | | 01/11/00 | | |
| | | | | 22/11/00 | | |
| | | | | N50 + A666 × 3 | | |
| | | | | 16/12/00 | | |
| | | | | N50 + A666 × 3 + V240 | | |
| | | | | 29/12/00 | | |
| 2 | M | 19 | Alc | Start:01/10/00 | | |
| | | | mod + inhalants + cannabis | | | |
| | | | | N50 + A666 × 3 + V240 | | |
| | | | inhalants major abuse | 08/10/00 | | |
| | | | drug | | | |
| | | | | 12/11/00 | | |
| | | | | 04/01/01 | | |

| | | | | | | Improved (1) Ancillary | | | |
|---|---|---|---|---|---|---|---|---|---|
| | Key Parameters | | | | | Parameters | | | Patient's |
| No | Audit Score | Craving | Relapse | Abstinence | Side-effects | Energy | Thinking | In-Control | View |
| 1 | 38 | 8 | 10 | 0 | | | | | |
| | 37 | 8 | 10 | 0 | Nil | 0 | 0 | 0 | 0 |
| | 37 | 7 | 8 | 1 | Nil | 0 | 0 | 1+ | Progress+ |
| | 35 | 6 | 5 | 4 | Nil | 0 | 0 | 1+ | Progress+ |
| | 33 | 4 | 3 | 7 | Nil | 0 | 0 | 1++ | Progress++ |
| | 37 | 7 | 8 | 2 | Mod | 0 | 0 | 0 | 0 |
| | 33 | 4 | 0 | 10 | Nil | 1+ | 1+ | 1++ | Progress++ |
| 2 | | 9 | 10 | 0 | | | | | |
| | | 2 | 0 | 10 | Nil | 0 | 1+ | 1+ | Progress++ |
| | | 1 | 0 | 10 | Nil | 1+ | 1++ | 1+++ | Progress+++ |
| | | 0 | 0 | 10 | Nil | 1++ | 1++ | 1+++ | Progress+++ |

*N, A and Pl as per Example 6

Sources of Drugs Used in the Treatments in Examples 6 and 7

Naltrexone (Revia^{R}, Orphan Australia Pty Ltd)—this drug is manufactured for Orphan by Du Pont Pharmaceuticals Company USA).

Amlodipine (Norvasc^{R}, Pfizer Australia Ltd).

Felodipine_{ER} (Plendyl^{R}, AstraZeneca Pty Ltd). Verapamil_{SR} (Isoptin^{R}, Knoll Australia Pty Ltd).

The claims:

1. A method of treating substance addiction in a subject in need thereof, which method comprises administering to said subject a combination of:
(i) a μ-opioid receptor antagonist (μORA);
(ii) a calcium channel blocker (CCB) which is long-acting or in sustained-release form, or which is nimodipine in rapid release form; and
(iii) an NMDA glutamate receptor modulator.

2. A method according to claim 1 wherein the μ-opioid receptor antagonist is selected from the group consisting of naltrexone, nalmefine, buprenorphine and 1-α-acetylmethadol.

3. A method according to claim 2 wherein the μ-opioid receptor antagonist is naltrexone.

4. A method according to claim 1 wherein the NMDA glutamate receptor modulator is selected from the group consisting of: CCP, dizocilpine, HA966, ibogaine, memantine; ifenprodil, eliprodil and acamprosate.

5. A method according to claim 4 wherein the NMDA glutamate receptor modulator is acamprosate.

6. A method according to claim 1 wherein the calcium channel blocker is selected from the group consisting of nifedipine, nimodipine, nisoldipine, felodipine, amlodipine, darodipine, floridipine, lacidipine, isradipine, niguldipine, niludipine, oxadipine, elgodipine, riodipine, nilvadipine, lemdipine, nitrendipine, nicardipine, verapamil; diltiazem and flunarizine.

7. A method according to claim 6 wherein the calcium channel blocker is selected from the group consisting of long-acting amlodipine and sustained-release verapamil, nifedipine and felodipine.

8. A method according to claim 1 wherein one or more of components (i)-(iii) are adapted for oral administration.

9. A method according to claim 3 wherein the naltrexone is administered in a once daily dose of 25 or 50 mg.

10. A method according to claim 5, wherein the acamprosate is administered as a thrice daily dose of 333 or 666 mg or a once or twice daily dose of about 1000 mg.

11. A method according to claim 6 wherein verapamil is administered in a once daily dose in the range of 80-480 mg.

12. A method according to claim 6 wherein amlodipine is administered in a once daily dose in the range of 2.5-20 mg.

13. A method according to claim 6 wherein felodipine is administered in a once daily dose in the range of 2.5-20 mg.

14. A method according to claim 6 wherein nifedipine is administered in a once daily dose in the range of 15-120 mg.

15. A method according to claim 6 wherein nitrendipine is administered in a once daily dose in the range of 5-20 mg.

16. A method according to claim 6 wherein the nisoldipine is administered in a once daily dose of 20-80 mg or a twice daily dose of 10-40 mg.

17. A method according to claim 6 wherein the nicardipine is administered in a once daily dose in the range of 30-120 mg or a twice daily dose of 15-60 mg.

18. A method according to claim 6 wherein the lacidipine is administered in a once daily dose in the range of 2-8 mg.

19. A method according to claim 6 wherein the diltiazem is administered in a once daily dose in the range of 90-360 mg.

20. A method according to claim 6 wherein the 15-60 mg nimodipine is administered 3 or 4 times daily.

21. A method according to claim 6 wherein the isradipine is administered in a once daily dose in the range of 2.5-20 mg.

22. A method according to claim 6 wherein 10-20 mg flunarizine is administered 1 time, 2 times or 3 times daily.

23. A method according to claim 1 wherein the substance of addiction is alcohol or a solvent inhalant or a combination of alcohol and one or more other addictive substances such as nicotine, an opiate or a solvent inhalant.

24. A method according to claim 1 which comprises the administration of a combination of naltrexone, acamprosate and verapamil.

25. A method according to claim 1 which comprises the administration of a combination of naltrexone, acamprosate and amlodipine.

26. A method according to claim 1 which comprises the administration of a combination of naltrexone, acamprosate and felodipine.

27. A method according to claim 1 which comprises the administration of a combination of naltrexone, acamprosate and nifedipine.

28. A method according to claim 1 which comprises the administration of a combination of naltrexone, acamprosate and nisoldipine.

29. A method according to claim 1 which comprises the administration of a combination of naltrexone, acamprosate and nitrendipine.

30. A method according to claim 1 which comprises the administration of a combination of naltrexone, acamprosate and nicardipine.

31. A method according to claim 1 wherein the substance of addiction is nicotine and the combination further comprises at least one of a ganglion nicotinic receptor antagonist, such as mecamylamine; or a nicotinic cholinergic receptor antagonist, such as bupropion; or γ-vinylGABA (vigabactin) or a κ-opioid agonist.

32. A composition for use in the method of claim 1 comprising at least two of:
(i) a μ-opioid receptor antagonist;
(ii) a calcium channel blocker which is long-acting or in sustained-release form, or which is nimodipine in rapid release form; and
(iii) an NMDA glutamate receptor modulator.

33. A kit for use in the method of claim 1 comprising at least two of:
(i) a μ-opioid receptor antagonist;
(ii) a calcium channel blocker which is long-acting or in sustained-release form, or which is nimodipine in rapid release form; and
(iii) an NMDA glutamate receptor modulator.

34. Use of a μORA in the preparation of a medicament for the treatment of substance addiction, wherein said medicament is adapted for administration to a subject in combination with an NMDA glutamate receptor modulator and a CCB, which is long-acting or in sustained-release form or which is nimodipine in rapid release form.

35. Use of a CCB which is long-acting or in sustained-release form, or which is nimodipine in rapid release form, in the preparation of a medicament for the treatment of substance addiction, wherein said medicament is adapted for administration in combination with a μORA and an NMDA glutamate receptor modulator.

36. Use of an NMDA glutamate receptor modulator in the preparation of a medicament for the treatment of substance addiction where said medicament is adapted for administration in combination with a CCB, which is long-acting or in sustained-release form, or which is nimodipine in rapid release form, and a μORA.

37. Use of a μORA, an NMDA glutamate receptor modulator and a CCB in the preparation of a medicament for the treatment of substance addiction, wherein the CCB is long-acting or formulated in the medicament such that it is in a sustained-release form or is nimodipine in rapid release form.
